(19) United States
(12) Patent Application Publication
(10) Pub. No.: US 2022/0233765 A1
(43) Pub. Date: Jul. 28, 2022

(54) CONDUCTIVE ELEMENT IN ADHESIVE PATCH OF ON-BODY DRUG DELIVERY DEVICE FOR BODY CHANNEL COMMUNICATION

(71) Applicant: Insulet Corporation, Acton, MA (US)

(72) Inventors: David NAZZARO, Groveland, MA (US); John D'ARCO, Wilmington, MA (US); Nicholas CONTE, Harvard, MA (US); Kepei SUN, Andover, MA (US)

(21) Appl. No.: 17/580,249

(22) Filed: Jan. 20, 2022

Related U.S. Application Data
(60) Provisional application No. 63/141,533, filed on Jan. 26, 2021.

(51) Int. Cl. A61M 5/142

(52) U.S. Cl. CPC A61M 5/14248; A61M 2205/3584; A61M 2205/0233; A61M 2210/04

(57) ABSTRACT

In an exemplary embodiment, a drug delivery device is configured for Body Channel Communication (BCC). The drug delivery device may include a conductive element embedded in an adhesive patch that secures the drug delivery device to the user. This conductive element acts as a coupler to the body of the user. Another conductive element may also be provided at a face of the housing. This conductive element acts as the other end of the coupler formed with the conductive coil in the adhesive patch. The conductive coil in the adhesive patch conforms well to the user's skin surface and thus facilitates a good quality coupling with the user.

### RELATED APPLICATIONS

This application claims the benefit of U.S. Provisional Patent Application No. 63/141,533, filed Jan. 26, 2021, the contents of which are incorporated herein by reference in their entirety.

### BACKGROUND

The radio frequency spectrum is divided into bands reserved for various uses. As the name implies, the Industrial, Scientific and Medical (ISM) band is reserved for non-telecommunications use. The ISM band is used for medical applications to facilitate wireless communication with medical devices, including on-body medical devices.

Unfortunately, there is increasing congestion of the ISM band. Microwave ovens operate at 2.45 GHz, which is in the ISM band. In addition, computers, printers and cellular phones often have IEEE 802.11 wireless modems that operate at 2.4 GHz in the ISM band. This congestion has made it increasingly difficult to use the ISM band for communications with medical devices, like drug delivery devices, that communicate at 2.4 GHz. The coexistence of so many communications in the ISM band can result in interference, noise and the like. In addition, 2.4 GHz communication with wearable medical devices often encounter significant energy absorption by the body.

### SUMMARY

In accordance with an inventive aspect, a capacitive coupling arrangement is provided for wireless transmission and reception via body channel communication for a drug delivery device. The capacitive coupling arrangement includes an electrically conductive surface on the exterior or interior face of a mechanical housing for the drug delivery device and an electrically conductive patch secured to or embedded in an adhesive patch attached to the bottom surface of the housing. The adhesive patch is for securing the drug delivery device to skin of the user. The electrically conductive patch is wirelessly connected to an electronic component in the drug delivery device. The electrically conductive patch couples the drug delivery device with the user's body for communication purposes.

The electrically conductive patch may be a sheet of metal. The electrically conductive patch may be a woven sheet. The electrically conductive patch may be a copper foil, aluminum foil or a silver foil, for example. The electrically conductive surface may be a conductive coating. The electrically conductive surface may be another conductive patch that is secured to the exterior face or interior face of the housing. The electrically conductive patch may be sufficiently flexible to conform to a curvature of the skin surface of the user when secured to the user.

In accordance with an inventive aspect, a drug delivery device has a top housing with an electrically conductive surface on an exterior face or an interior face of a surface of the housing. The drug delivery device has a bottom housing that forms a complete housing when interconnected with the top housing. The drug delivery device further includes an adhesive patch secured to a bottom face of the bottom housing. The patch has an adhesive for securing the drug delivery device to a skin surface of a user. The drug delivery device additionally includes an electronic component and an electrically conductive patch secured to, embedded in, or surrounded by the adhesive patch on the bottom face of the bottom housing for electrically coupling the drug delivery device with a body of the user. The electrically conductive patch couples to the electronic component via a conductive surface on its exterior face or the interior face of its top housing thus forming a capacitive coupling arrangement for wireless transmission and reception via body channel communication for the drug delivery device.

The drug delivery device may be an insulin pump, a glucagon pump, or another therapeutic agent or drug pump, or a combination thereof. The electronic component may be a wireless communication transceiver. The conductive patch may be a sheet of metal. The electrically conductive patch may be a copper foil, aluminum foil or a silver foil. The electrically conductive patch may be a woven sheet. The electrically conductive surface may be a conductive coating. The electrically conductive surface may be another conductive patch which is secured to the exterior face or the interior face of the housing. The electrically conductive patch may be sufficiently flexible to conform when secured to the curvature of the user's skin.

In accordance with an inventive aspect, a method includes providing an electronic component in a drug delivery device that is configured to be worn on the body of a user. The method also entails creating an electrically conductive surface on an exterior face of a housing of the drug delivery device or on interior face of the housing and securing an electrically conductive patch over a bottom face of a bottom housing of the drug delivery device for electrically coupling the drug delivery device with the body of the user. The method further includes electrically connecting the conductive patch with the electronic component so that the electrically conductive patch and the electrically conductive surface on the exterior face or the interior face of the housing form a capacitive coupling arrangement for wireless transmission and reception via body channel communication (BCC) for the drug delivery device.

Securing an electrically conductive patch over a bottom face of a housing of the drug delivery device may include embedding the electrically conductive patch in an adhesive patch, surrounding the electrically conductive patch with an adhesive patch, or securing the electrically conductive patch to an adhesive patch, and securing the adhesive patch to the body of the user. Creating an electrically conductive surface on a top face of a top housing of the drug delivery device may entail securing a metal foil to an exterior face of the housing or on the interior face of the housing.

### BRIEF DESCRIPTION OF THE DRAWINGS

FIG. 1 depicts an illustrative drug delivery system for an exemplary embodiment.

FIG. 2A depicts an illustrative drug delivery device of an exemplary embodiment and a depiction of corresponding electric fields that may be produced in using the drug delivery device for BCC.

FIG. 2B-2E depicts illustrative arrangements of the signal electrode and adhesive pad for exemplary embodiments

FIG. 3 depicts a diagram of different types of conductive patches that may be used in exemplary embodiments.

FIG. 4 depicts a flowchart of illustrative steps that may be performed in transmitting information from the drug delivery device via BCC in an exemplary embodiment.

FIG. 5 depicts a flowchart of illustrative steps that may be performed in receiving information via BCC from the drug delivery device in an exemplary embodiment.

### DETAILED DESCRIPTION

Exemplary embodiments may overcome the problems encountered with conventional communications with medical devices by using Body Channel Communication (BCC) for communications with medical devices. Exemplary embodiments may provide an on-body drug delivery device that is configured for BCC. By communicating using BCC, the on-body drug delivery device does not encounter the difficulties associated with congestion as found with conventional communication in the ISM band. BCC uses frequencies below 2.4 GHz, or more particularly, frequencies below 100 MHz, or between 100 kHz and 150 MHz. In addition, BCC does not suffer from the problem of significant energy absorption that is encountered with conventional wireless communication with medical devices at 2.4 GHz.

In an exemplary embodiment, the drug delivery device is configured for BCC. As will be explained a coupler is formed by two electrically conductive elements. These elements form the plates of a capacitive coupler. The capacitive coupler may be used for transmission and receipt of communications via magnetic or electrical fields. The drug delivery device may include a conductive element secured to, embedded in, or surrounded by an adhesive patch that secures the drug delivery device to the user. The conductive element may be a metal foil, a woven sheet or other type of electrically conductive element. This conductive element acts as a coupler to the body of the user. A conductive element, like a metal foil, woven sheet or other conductive surface or patch, may also be provided at an exterior face or an interior face of the housing. This conductive element acts as the other end of the coupler formed with the conductive element in the adhesive patch. The conductive foil in the adhesive patch is configured to conform well to the skin surface of the user and thus facilitates good quality coupling with the user.

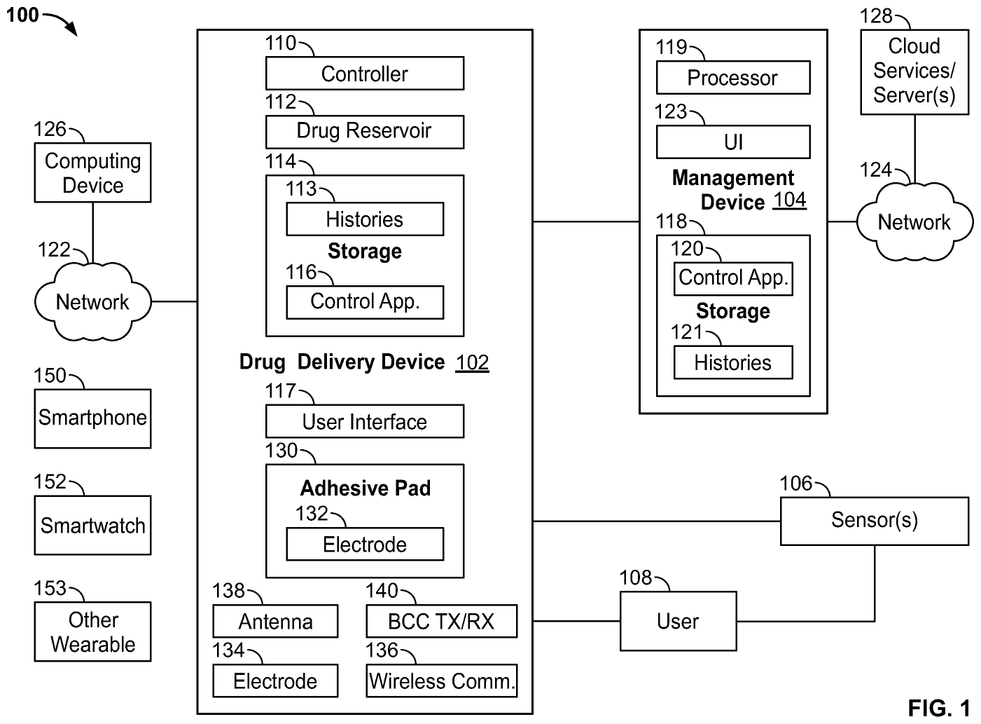

FIG. 1 depicts an illustrative drug delivery system (100) that includes an on-body drug delivery device (102) having components for communicating via BCC. The on-body drug delivery device (102) may be directly coupled to a user. In an example, a surface of the on-body drug delivery device (102) may include an adhesive pad (130) to facilitate attachment to the user (108).

The on-body drug delivery device (102) may include a controller (110). The controller (110) may be implemented in hardware, software, or any combination thereof. The controller (110) may, for example, be a microprocessor, a logic circuit, a field programmable gate array (FPGA), an application specific integrated circuit (ASIC) or a microcontroller coupled to a memory. The controller (110) may maintain a date and time as well as other functions (e.g., calculations or the like). The controller (110) may be operable to execute a control application (116) stored in the storage (114) that enables the controller (110) to direct operation of the on-body drug delivery device (102) and a processor within controller (110) may execute processes to manage a user's blood glucose levels by controlling delivery of the drug or therapeutic agent to the user (108). The storage (114) may hold histories (113) for a user. Where the on-body drug delivery device (102) is an insulin delivery device, the histories (113) may include information such as a history of automated insulin deliveries, a history of bolus insulin deliveries, meal event history, exercise event history and the like. In addition, the controller (110) may be operable to receive data or information, such as signals from sensor (106) or user commands from management device (104). The storage (114) may include both primary memory and secondary memory. The storage (114) may include random access memory (RAM), read only memory (ROM), optical storage, magnetic storage, removable storage media, solid state storage or the like.

The on-body drug delivery device (102) may include a drug reservoir (112) for storing a drug for delivery to the user (108) as warranted. A fluid path to the user (108) may be provided, and the on-body drug delivery device (102) may expel the drug from the drug reservoir (112) to deliver the drug to the user (108) via the fluid path. In an exemplary embodiment, the drug may be insulin. The fluid path may, for example, include tubing coupling the on-body drug delivery device (102) to the user (108) (e.g., tubing coupling a cannula to the drug reservoir (112)).

There may be one or more communications links with one or more devices physically separated from the on-body drug delivery device (102) including, for example, a management device (104) of the user and/or a caregiver of the user, a sensor for sensing an analyte (106) and/or an adhesive pad (130). The analyte being sensed may be blood glucose concentration, lactate, ketones, sodium, potassium, uric acid, alcohol levels, drug concentrations, or the like. A wireless transceiver (136) may be provided to transmit and receive wireless communications, such as Bluetooth®, Bluetooth® Low Energy (BLE), IEEE 802.11, Zigbee or Wireless Body Area Network (WBAN) (IEEE 801.15.6) communications. An antenna (138) may be provided for such communications. Such communications are well adapted for off-body communications. This antenna (138) may be realized as a slot antenna formed in a slot between the signal electrode (132) in the adhesive pad (130) and the electrode 134 formed on the top portion of the housing.

As mentioned above, the on-body drug delivery device is configured for BCC communications. To that end, an electrode (132) is provided that is secured to, embedded in, or surrounded by the adhesive pad 130. In addition, another electrode (134) is provided to complete a coupler for BCC communications as will be described below. The electrode (134) may be secured to another adhesive patch, formed by conductive coating or otherwise formed on the top or side portion of the housing of the on-body drug delivery device (102). A BCC transceiver (140) is provided for transmitting signals via the coupler and receiving signals via the coupler as will be described in more detail below. The on-body drug delivery device (102) may also include a user interface (117), such as an integrated display device for displaying information to the user (108) and in some embodiments, receiving information from the user (108). The user interface (117) may include a touchscreen and/or one or more input devices, such as buttons, a knob or a keyboard.

The on-body drug delivery device (102) may interface with a network (122). The network (122) may include a local area network (LAN), a wide area network (WAN) or a combination therein. A computing device (126) may be interfaced with the network, and the computing device may communicate with the on-body drug delivery device (102).

The drug delivery system (100) may include the sensor (106) for sensing one or more analytes as discussed above (108). In some exemplary embodiments, the sensor (106) may provide periodic blood glucose concentration measurements and may be a continuous glucose monitor (CGM), or another type of device or sensor that measures analytes as described above. The sensor (106) may be physically separate from the on-body drug delivery device (102) or may be an integrated component thereof. The sensor (106) may provide the controller (110) with data indicative of measured or detected blood glucose levels of the user (108) in some exemplary embodiments. The sensor (106) may be coupled to the user (108) by, for example, adhesive or the like and may provide information or data on one or more medical conditions and/or physical attributes of the user (108). The information or data provided by the sensor (106) may be used to adjust drug delivery operations of the on-body drug delivery device (102) by the on-body drug delivery device (102) alone or with additional input (e.g., user input) from management device 104. The on-body drug delivery device (102) may communicate with the sensor (106) using the coupler for BCC communications. The BCC coupler described herein is well suited for communicating with on-body medical devices.

The drug delivery system (100) may also include management device (104). The management device (104) may be a special purpose device, such as a dedicated personal diabetes manager (PDM) device. Alternatively, the management device (104) may be a programmed general-purpose device, such as any portable electronic device including, for example, a dedicated controller, a smartphone, a smartwatch, a tablet, or a combination thereof. The management device (104) may be used to program or adjust operation of the on-body drug delivery device (102) and/or the sensor (106). In the depicted example, the management device (104) may include a processor (119) and a storage (118). In some exemplary embodiments, the processor (119) may execute processes to manage a user's blood glucose levels by controlling the delivery of the drug or therapeutic agent to the user (108). The processor (119) may also be operable to execute programming code stored in the storage (118). For example, the storage may be operable to store one or more control applications (120) for execution by the processor (119). The storage (118) may store the control application (120), histories (121) like those described above for the insulin delivery device (102) and other data and/or programs.

The management device (104) may include a user interface (123) for communicating with the user (108). The user interface may include a display, such as a touchscreen, for displaying information. The touchscreen may also be used to receive input when it is a touch screen. The user interface (123) may also include input elements, such as a keyboard, buttons, knobs, or the like.

The management device (104) may interface with a network (124), such as a LAN or WAN or combination of such networks. The management device (104) may communicate over network (124) with one or more servers or cloud services (128).

The drug delivery system (100) may also include other external devices, such as a smartphone (150), a smartwatch (152) or wearable health, biometric or therapeutic devices (153). The on-body drug delivery device (102) may communicate with the smartphone (150) or smartwatch (152) via BCC if these components (150) and (152) are configured to include a coupler as needed for BCC. For example, conductive elements as described below may be provided on the top and bottom portions of the smartphone (150), smartwatch (152) and BCC transceiver (140) as is described herein. The smartphone (150), the smartwatch (152) or the other wearable (153) may use BCC communications to receive data to display and to perform management operations that are communicated to the on-body drug delivery device (102) via BCC. Such BCC communications may also take place between the on-body drug-delivery device (102) and the management device (104) or the sensor (106) if those components (104) and (106) are configured for such communications by having conductive elements, a BCC transceiver (140), etc. as described herein. BCC provides superior communication between on-body devices relative to conventional wireless communications that suffer from interference, poor directivity and signal loss due to body absorption as described above.

With BCC, the human body is used as the transmission medium for electrical signal transfer. In one form of BCC, capacitive coupling is used. With capacitive coupling, electric fields are used for signal transmission. A signal electrode is secured to the skin of the user and delivers or receives a signal. The signal is delivered by forming an electric field induced by the signal electrode. A signal electrode may also be used for receiving a signal from the body of the user.

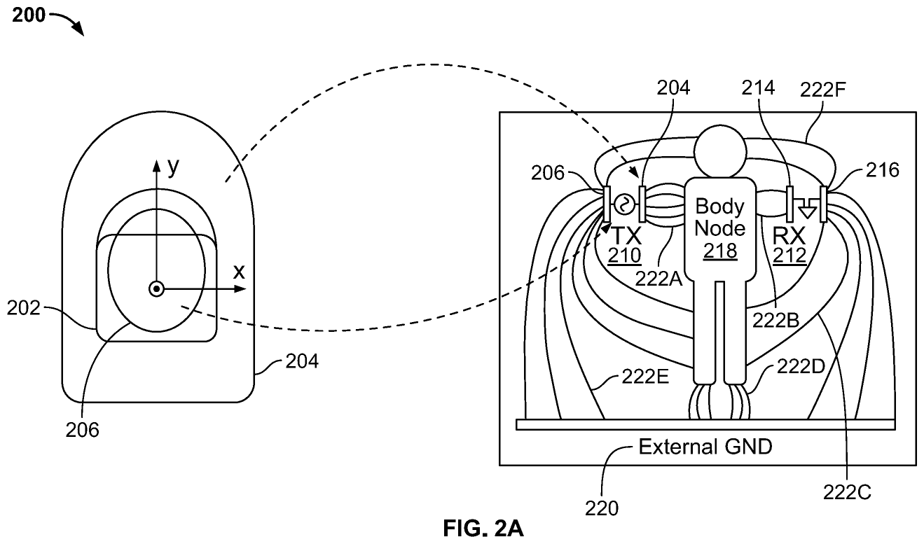

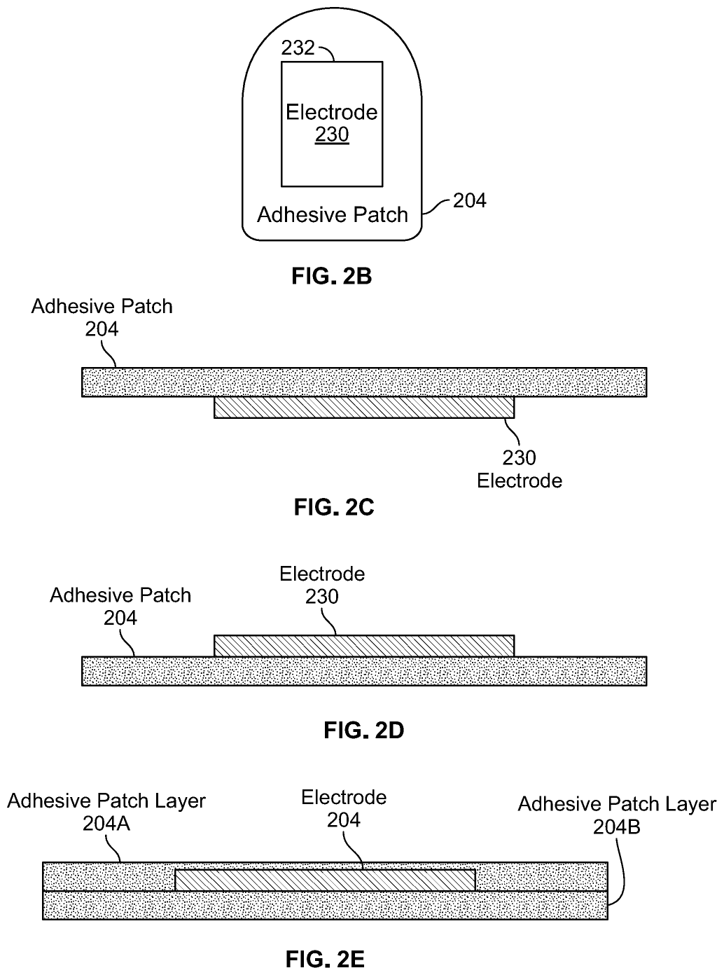

FIG. 2A depicts an illustrative on-body drug delivery device (200) for an exemplary embodiment. In this example, the on-body drug delivery device (200) is an insulin pump. The on-body drug delivery device (200) includes a housing (202) for housing components such as a drug reservoir (112), storage (114), user interface (117), wireless communication transceiver (136), antenna (138) and BCC transceiver (140) shown in FIG. 1. The adhesive pad (130) includes a conductive patch (204) for a signal electrode (230). The signal electrode (230) may be woven into the adhesive pad (130), may be surrounded by the adhesive pad (130), or may be secured to the top or bottom of the adhesive pad (130) via adhesive or other securing mechanism.

FIG. 2B shows an example where the signal electrode (230) is secured in the adhesive patch (204) as described above. A cutout (2320 in the adhesive patch (204) exposes the signal electrode (230) so that the signal electrode (230) may contact the skin surface of a user. The signal electrode (230) alternatively may be secured on the underside of the adhesive patch (204) as shown in FIG. 2C. For example, the adhesive on the underside of the adhesive patch (204) may hold the signal electrode in place and secure the adhesive patch (204) to the skin surface of the user. The signal electrode (230) instead may be secured to the top surface of the adhesive patch (204) as mentioned above and as depicted in FIG. 2D. Further, the signal electrode (230) may be secured in the adhesive patch (204), such as between adhesive patch layers (204A) and (204B) as shown in FIG. 2E.

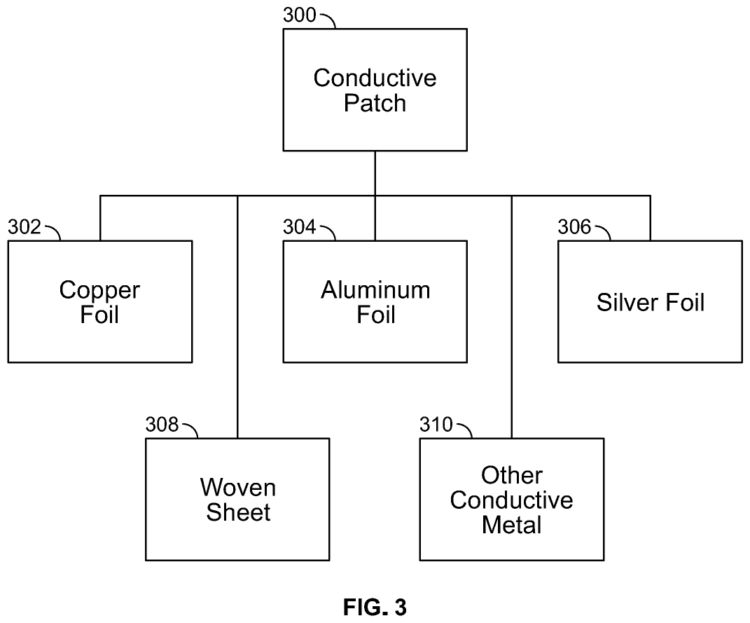

The conductive patch (300) may take many different forms, as shown in FIG. 3. In some exemplary embodiments, the conductive patch is a metallic foil (i.e., a thin sheet of metal). For instance, the conductive patch (300) may be a copper foil (302), an aluminum foil (304) or a silver foil (306). Alternatively, the conductive patch may be a woven sheet (308) having conductive metal components. More generally, the conductive patch (300) may be another type of conductive metal (310) in a foil form or in another form that enables the conductive patch to act as an electrode. The conductive patch (300) forms the signal electrode (132) on the adhesive pad (130) that is secured to skin of the user. The conductive patch (300) may be flexible so as to conform with the contours of the skin surface and provide good quality coupling.

The on-body drug delivery device (102) also includes a conductive surface (206) on a top region of the housing (202). More particularly, the conductive surface (206) may be positioned, for example, on the inside of the top of the housing, the outside of the top housing, on the outer or inner surfaces of the sides of the housing (202). More generally, the conductive surface (206) may be place at other locations in or on the drug delivery device. This conductive surface (206) may be another conductive patch like that previously described, may be a conductive coating applied to the inner surface of the housing (202), or generally may be formed by deposit, printing or other means to an interior surface or exterior surface of the top or side portions of the housing (202). The conductive surface (206) acts as a floating ground electrode. The floating electrode is coupled to ground (220) via air, creating a return path. The conductive foil or element in the conductive patch (204) on or in or adjacent the adhesive pad serves as the signal electrode (230) that transmits a signal via the user's body using Body Channel Communication at a frequency lower than 2.4 GHz, such as 100 kHz to 150 MHz

FIG. 2A shows the transmitter (210) and the receiver (212) in a BCC arrangement. The transmitter (210) and the receiver (212) are each capacitive couplers having capacitive plates as will be described below. Each capacitive coupler (210) and (212) may be part of a respective on-body medical device, such as the on-body drug delivery device (200) described herein, and the capacitive couplers (210) and (212) are used for BCC wireless communications. In this example, it is assumed that the on-body drug delivery drug delivery device (200) contains the transmitter (210). The transmitter (210) has the conductive surface (206) on the inner (e.g., top) surface of the housing (202) acting as a ground plane and the signal electrode (230) on a bottom surface of the housing (e.g, secured to or embedded in the adhesive) . The dotted lines indicate the mapping of these elements to the transmitter 210.

The transmitter (210) generates an electric potential so that electric fields are induced from the signal electrode in the conductive patch. Electric field lines (222A) indicate electric fields that are transmitted through the body (218) of the user originating from the signal electrode (230). Some of these electric fields (222B) are received by a signal electrode (214) attached to the skin of the user and forming part of the coupler of the receiver (212). As indicated above, the receiver (212) is a coupler that is part of another on-body medical device. The depiction also shows additional electric field lines (222C), (222D), (222E) and (222F). The depiction further shows the external ground (220).

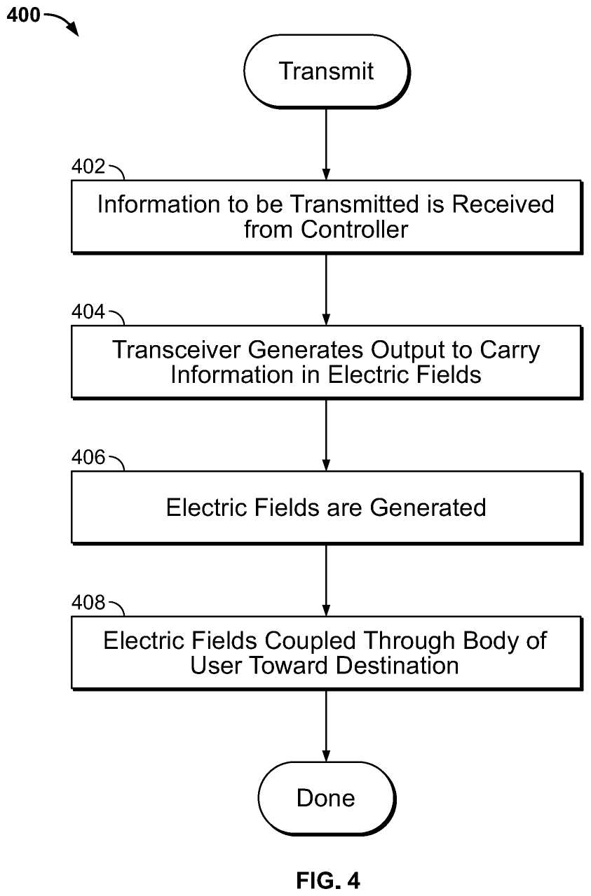

FIG. 4 depicts a flowchart (400) of illustrative steps that may be performed in transmitting a signal from the drug delivery device (102) in an exemplary embodiment. Initially, at 402, the information to be transmitted is received by the BCC transceiver (140) from the controller (112). This information may be a message, a data packet, or the like. The transceiver generates an output to carry the information by generation of an electric field at 404. As was mentioned above, at 406, an electric potential is created that induces the electric field using the transmitting signal electrode (204) that is attached to the skin of the user (218). The electric field, in part, is directed to the body (218) of the user, as indicated by electric field lines 222A. The electric fields are coupled through the body (218) of the user toward the desired destination.

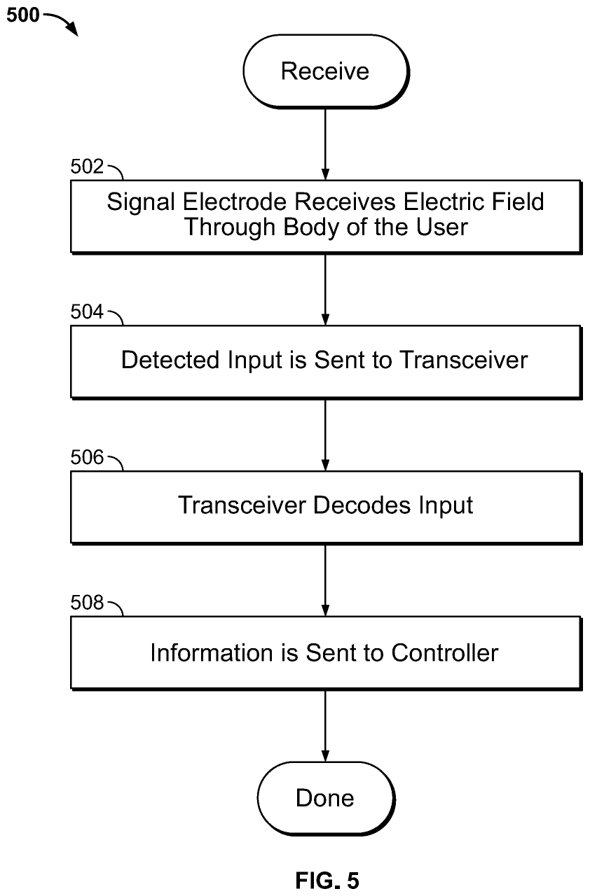

FIG. 5 depicts a flowchart (500) of illustrative steps that may be performed in receiving a signal by the drug delivery device (102). At 502, the transmitted electric field travels through the body (218) of the user and is received by a receiver signal electrode (214). As can be seen in FIG. 2, electric field lines (222B) represent the electric field transmitted by the transmitter (210) that has traveled through the body (218) of the user and which is received by the receiver signal electrode (214). At 504, the receiver signal electrode forwards the detected input to the BCC transceiver (140). At 506, the BCC transceiver (140) decodes the received input and extracts the information encoded in the received input. At 508, the resulting extracted information is sent to the controller (110).

While exemplary embodiments have been described herein, it will be appreciated that various changes in form and detail may be made without departing from the scope of claims appended hereto.

1. A capacitive coupling arrangement for wireless transmission and reception via body channel communication for a drug delivery device, comprising:
an electrically conductive surface on an exterior face or an interior face of a housing surface for the drug delivery device;
an electrically conductive patch secured to or embedded in an adhesive patch secured to a bottom surface of the housing, wherein:
the adhesive patch is for securing the drug delivery device to skin of the user;
the electrically conductive patch is electrically connected to an electronic component in the drug delivery device; and
the electrically conductive patch is for electrically coupling the drug delivery device with a body of the user.

2. The capacitive coupling arrangement of claim 1, wherein the electrically conductive patch is a sheet of metal.

3. The capacitive coupling arrangement of claim 1, wherein the electrically conductive patch is a woven sheet.

4. The capacitive coupling arrangement of claim 1, wherein the electrically conductive patch is a copper foil, aluminum foil or a silver foil.

5. The capacitive coupling arrangement of claim 1, wherein the electrically conductive surface is a conductive coating.

6. The capacitive coupling arrangement of claim 1, wherein the electrically conductive surface is another conductive patch that is secured to the exterior face or the interior face of the housing surface.

7. The capacitive coupling arrangement of claim 1, wherein the electrically conductive patch is sufficiently flexible to conform to a curvature of the skin surface of the user when secured to the user.

8. A drug delivery device, comprising:
a top housing having an electrically conductive surface on an exterior face or an interior face of the top housing;
a bottom housing that forms a complete housing when interconnected with the top housing;
an adhesive patch secured to a bottom face of the bottom housing, the adhesive patch having an adhesive for securing the drug delivery device to a skin surface of a user;
an electronic component;
an electrically conductive patch secured to or embedded in the adhesive patch on a bottom face of the bottom housing for electrically coupling the drug delivery device with a body of the user, wherein the electrically conductive patch is electrically connected to the electronic component and the electrically conductive patch and the electrically conductive surface on the exterior or interior face of the top housing form a capacitive coupling arrangement for wireless transmission and reception via body channel communication for the drug delivery device.

9. The drug delivery device of claim 8, wherein the drug delivery device is an insulin pump, glucagon pump, a therapeutic agent pump or a combination thereof.

10. The drug delivery device of claim 8, wherein the electronic component is a wireless communication transceiver.

11. The drug delivery device of claim 8, wherein the conductive patch is a sheet of metal.

12. The drug delivery device of claim 8, wherein the electrically conductive patch is a copper foil, aluminum foil or a silver foil.

13. The drug delivery device of claim 8, wherein the electrically conductive patch is a woven sheet.

14. The drug delivery device of claim 8, wherein the electrically conductive surface is a conductive coating.

15. The drug delivery device of claim 8, wherein the electrically conductive surface is another conductive patch that is secured to the exterior or interior face of the housing.

16. The drug delivery device of claim 8, wherein the electrically conductive patch is sufficiently flexible to conform to a curvature of the skin surface of the user when secured to the user.

17. A method, comprising:
providing an electronic component in a drug delivery device that is configured to be worn on a body of a user;
creating an electrically conductive surface on an exterior face or an interior face of a housing of the drug delivery device;
securing an electrically conductive patch over a bottom face of the housing of the drug delivery device for electrically coupling the drug delivery device with the body of the user; and
electrically connecting the electrically conductive patch with the electronic component so that the electrically conductive patch and the electrically conductive surface on the exterior or interior face of the housing form a capacitive coupling arrangement for wireless transmission and reception via body channel communication for the drug delivery device.

18. The method of claim 17, wherein the securing an electrically conductive patch over a bottom face of the housing of the drug delivery device comprises embedding the electrically conductive patch in an adhesive patch and securing the adhesive patch to the body of the user.

19. The method of claim 17, wherein the securing an electrically conductive patch over a bottom face of the housing of the drug delivery device comprises securing the electrically conductive patch to an adhesive patch and securing the adhesive patch to the body of the user.

20. The method of claim 17, wherein the creating an electrically conductive surface on the exterior face or the interior face of a housing of the drug delivery device comprises securing a metal foil to the exterior or interior face of the housing.
